(19) United States
(12) Patent Application Publication
(10) Pub. No.: US 2013/0164336 A1
(43) Pub. Date: Jun. 27, 2013

(54) ANTIMICROBIAL AND ANTI-INFLAMMATORY ACTION PHARMACEUTICAL COMPOSITION FOR PARENTERAL ADMINISTRATION AND ITS PRODUCTION PROCESS

(71) Applicants: Viktor Lvovich Limonov, Moscow (RU); Konstantin Valentinovich Gaidul, Novosbirsk (NO); Aleksandr Valerevich Dushkin, Novosbirsk (RU)

(72) Inventors: Viktor Lvovich Limonov, Moscow (RU); Konstantin Valentinovich Gaidul, Novosbirsk (NO); Aleksandr Valerevich Dushkin, Novosbirsk (RU)

(21) Appl. No.: 13/389,533

(22) PCT Filed: May 11, 2011
(86) PCT No.: PCT/RU11/00321

§ 371 Date: Feb. 8, 2012

(30) Foreign Application Priority Data
Sep. 20, 2010 (EP) EA201001507

(51) Int. Cl. A61K 9/14; A61K 31/665

(52) U.S. Cl. CPC A61K 9/143; A61K 31/665
USPC 424/400; 514/99

(57) ABSTRACT

There is proposed herein a process for production of composite antimicrobial preparations for parenteral administration, featuring a higher therapeutic efficiency in case of grave infection and inflammatory diseases. The proposed compositions include an active agent being fosfomycin and finely dispersed nanostructured silica dioxide, with a weight ratio from 10:1 to 75:1 respectively. The mentioned production process includes mixing fosfomycin with finely dispersed nanostructured silica dioxide. Its main difference is that the mixture of aforementioned substances with the mentioned weight ratio is exposed to mechanical processing by blow impact and abrasive actions until a portion of the fine powder fraction with particles smaller than 5 micrometers, contained in the mixture, increases to at least 25%. The so obtained mixture is used for injection preparations.

### CROSS-REFERENCE TO RELATED APPLICATIONS

This application is a U.S. national stage application of a PCT application PCT/RU2011/000321 filed on 11 May 2011, whose disclosure is incorporated herein in its entirety by reference, which PCT application claims priority of a EAPO application EA201001507 filed on 20 Sep. 2010.

### FIELD OF THE INVENTION

This invention belongs to antimicrobial pharmaceutical preparations and its' production technologies. It can be used in medicine and veterinary science for treating contagious and inflammatory diseases, as well as being used in pharmaceutical industry for medicinal products manufacturing.

### BACKGROUND OF THE INVENTION

At the moment, to ensure a successful therapy of many contagious and inflammatory diseases (sepsis, meningitis, osteomyelitis, pyelonephritis and etc.) practicing physicians of different specializations are widely using an antibiotic with the international nonproprietary name—fosfomycin [1, 2, 3, 4, 5].

The mentioned antibiotic possesses a wide range antimicrobial action and provides a bacillicidal effect on many gram-positive and gram-negative microorganisms, which provoke contagious processes in different organs and tissues.

Microorganism's fosfomycin secondary resistance develops slowly, and the absence of cross-resistance with other antibiotics, allow to successfully combine it with betalactam and aminoglycosides for apparent synergy achievement [1, 6, 7].

Fosfomycin has a unique ability to inhibit the synthesis and proinflammatory cytokines (IL-1, IL-6, TNF and etc.) with human blood cells, which has a high clinical significance during generalized infections treatment and sepsis in particular [8].

Although taking into consideration all fosfomycin positive qualities it is necessary to bring into focus that its' monotherapy of contagious and inflammatory processes provoked by microorganisms with different sort of resistance to this antibiotic, is not always efficient. This fact makes many specialists to change fosfomycin to other antibiotics or makes them use a simultaneous injection of other antimicrobial agents [6, 9], which is highly unwanted from clinical and economical point of view.

That is why the finding new approaches to increase antimicrobial activity and clinical effectiveness of fosfomycin is a relevant objective of the modern experimental pharmacology and practical medicine.

In the past few years, it has been discovered that using different nano-particles as delivery agents for different antibiotics into bacterias and macrophages to increase their concentration at the inflammation area and consequently improve their antimicrobial characteristics, as well as simulate macrophages antibacterial activity and their additional recruitment into the infected tissues, is a very perspective tendency for original technologies and methods of antibiotic therapy development [10, 11, 12, 13, 14, 15, 16, 17, 18, 19].

### DESCRIPTION OF THE INVENTION

As a main point of this invention, it is suggested to use SiO₂ (silica dioxide) nano-particles which having pharmacologically profitable qualities of biocompatibility, biodistribution, biodegradation and low toxicity (not depending on the structure porosis intensity rate), are capable of serving as antibiotics endocellular delivery agents into macrophages in case of parenteral administration. These macrophages are concentrated in the areas of inflammation which can be seen in lungs, liver, kidneys, lien, lymph glands, heart, skin, urinary bladder and other organs of the mammals (i.e. considerably increase antibiotic concentration in the infected areas) as well as stimulate the antimicrobial activity of those immune system cells. This will lead to antimicrobial preparations therapeutic efficiency in case of contagious and inflammatory diseases treatment [20, 21, 22, 23, 24, 25, 26, 27].

The mentioned invention solves the issue of creating an antimicrobial and anti-inflammatory action pharmaceutical composition for injections on basis of using fosfomycin and silica dioxide nano-particles, which possess an increases therapeutic efficiency (comparing to the initial fosfomycin, which is considered as the prototype of the mentioned invention) in case of contagious and inflammatory diseases treatment.

To solve the assigned task it is suggested to use an antimicrobial and anti-inflammatory action pharmaceutical composition for parenteral administration, which contains fosfomycin injection powder as therapeutic substance as well as finely dispersed nanostructured silica dioxide with a weight ratio—fosfomycin:finely dispersed nanostructured silica dioxide w/w (10-70):1.

The portion of the finely dispersed nanostructured silica dioxide with the dimension ≦5 micron is not less than 25%.

To solve the assigned task it is also suggested to use an antimicrobial and anti-inflammatory action pharmaceutical composition for parenteral administration, produced by mixing fosfomycin with other components, i.e. mixing fosfomycin injection powder as therapeutic substance with finely dispersed nanostructured silica dioxide with a weight ratio—fosfomycin:finely dispersed nanostructured silica dioxide w/w (10-70):1. The received mixture is being mechanized by impact and abrasive actions.

The received mixture is being mechanized by impact and abrasive actions to make the portion of the finely dispersed nanostructured silica dioxide with the dimension ≦5 micron not less than 25%.

Therapeutic efficiency of the proposed pharmaceutical composition will increase, if it the received mixture is being mechanized by impact and abrasive actions to make the portion of the finely dispersed nanostructured silica dioxide with the dimension ≦5 micron not less than 25%.

To prepare the mentioned pharmaceutical composition, we used fosfomycin (parenteral administration form) produced by a Spanish company “Ercros”. As a finely dispersed nanopatterned silica dioxide (hereafter referred to as BHSiO₂) was used “Polysorb” drug (pharmacological group: enterosorbing solution; active substance: colloidal silica dioxide), produced by Russian company CJSC “Polysorb”, containing round shaped silica dioxide nanoparticles (dimension 5-20 nm) combined into aggregates (irregular microparticles) with dimension ≦90 micron (registration number No 001140/01-100908). There is s similar preparation produced by Ukrainian company CJSC “Biopharma” with a trade name “Silics” [24].

The composition formulation choice was based on convertible betalactam molecules and nano- as well as micro BHSiO₂ particles sorption process, together with BHSiO₂ particles reduction during its' mixtures mechanical activation with fosfomycin substances by impact abrasive mechanization process.

The stated production process of the previously mentioned pharmaceutical composition by fosfomycin powder mixture and BHSiO2 mechanical activation with intensive impact abrasive operations allows to increase the finely dispersed BHSiO2 particles (less than 5 micron) on which fosfomycin molecules are adsorbed and which are mostly phagocyted by macrophages [28].

To achieve this goal the mixture of the stated above materials in weight rating, fosfomycin:BHSiO₂ w/w (10-75):1, is exposed to intensive impact abrasive mechanical activation process until the finely divided fraction weight rating is increased up to 25%.

From the received powder like composition you can prepare an injection sol for parenteral insertion (water it down by any means appropriate for fosfomycin), composed of finely dispersed BHSiO₂ particles with inversibly sorbed any fosfomycin molecules on its surface.

Introducing of the finely dispersed nanostructured silica dioxide equal fosfomycin:BHSiO₂ from 10:1 to 75:1 regarding its' weight is determined by the combination of 2 factors: 1) during BHSiO₂ more than 10% increase from the composition weight in case of laboratory animals, they suffer from the small capillary tube blockage of solid viscus; 2) in case of BHSiO₂ content decrease for more than 1% of the composition weight (in particular during the mice treatment of bacterial sepsis) it's therapeutic efficiency doesn't differ from the initial antibiotic basic efficiency.

To receive the composition mechanochemical method was used, which comprehends the solid components mixture processing by intensive mechanical impacts—pressure and shearing deformations, mostly realized in different kind of mills which perform impact abrasive actions on the substances. The mixture of the solid fosfomycin substance and finely dispersed nanostructured silica dioxide taken in the ratio from 10:1 to 75:1 by weight, are exposed to bead mills mechanical activation. The used mixture preparation method helps in a certain way to avoid chemical degradation and achieve powder components full homogeneity in comparison with making the mixture by a simple components mixing, or evaporating their solutions, and as consequence causes a high pharmacologic activity of pharmaceutical composition.

As a quantitative criterion of the minimum necessary mechanical impact dose it is comfortable to use the granulometry method of the composition suspension. It is necessary that the mass fraction of the particles less than 5 micron was more than 25%. Powder mixtures mechanical processing is performed in rotary, vibrational and planetary mills. As grinding bodies you can use balls, cores and etc.

Laboratory animals (mice) pharmacological tests of the compositions showed, that the mentioned compositions prepared by the mentioned method have a higher therapeutic efficiency while treating bacterial sepsis, provoked by Staphylococcus aureus, Escherichia coli and Pseudomonas aeruginosa, comparing to the initial fosfomycin.

In such manner, using the mentioned pharmacological compositions and their production process provide the stated below advantages:
1) Clinically significant increase of the effectiveness and quality of the antimicrobial therapy of semi-acute and acute infection inflammatory diseases, death rate reduction;
2) Ecological safety, lack of wastes and low price of pharmacological production technology.
   The proposed invention is illustrated by examples listed below.

### Example No 1

### Solid Composition Production: Fosfomycin/ BHSiO₂

The mixture of fosfomicin and BHSiO₂ in weight ratio 10:1, 30:1 and 50:1 is being processed for 1, 2 or 4 hours in a rotary mill. In table No 1 you will see the granulometric composition data for the water suspensions of the received compositions variations (Micro-Sizer 201 laser granulometer was used) as well as HELC analysis results which show their antibiotics content (in % from the initial substance) and fosfomicin sorption degree (in %) by BHSiO₂ particles,

As can be seen from Table No 1 the chosen conditions of the composition production afford to increase until a certain value (not less than 25% from the total weight) the part of the finely dispersed BHSiO₂ fraction (particles size less than 5 micron) and to avoid the antibiotic chemical degradation.

TABLE NO1
Water suspensions granulometric composition,
fosfomycin sorption rate and content in different composition variations

| | Dimension | | | |
|---|---|---|---|---|
| | and content | | Fosfomycin | |
| | % of BHSiO₂ | | sorption rate | |
| | particles* | | by BHSiO₂ | Fosfomycin |
| | % < 3 | % < 5 | particles | content |
| Composition content | micron | micron | (%) | (%) |
| Initial BHSiO₂ | 0.5 | 5.7 | — | — |
| Fosfomycin:BHSiO₂ | 12.3 | 28.9 | 30.2 | 99 |
| (10:1), m/a 1 hour | | | | |
| Fosfomycin:BHSiO₂ | 17.6 | 31.8 | 41.1 | 99 |
| (30:1), m/a 2 hours | | | | |
| Fosfomycin: BHSiO₂ | 16.1 | 30.7 | 42.9 | 98 |
| (50:1), m/a 4 hours | | | | |

*finely dispersed nanostructured silica dioxide

### Example No 2

### Determination of the Therapeutic Efficiency of Fosfomycin and Pharmaceutical Compositions

There has been a research of fosfomycin mechanized for 2 hours and composed of a mixture antibiotic/BHSiO₂ in weight ratio 30:1 and 50:1 respectively.

To determine therapeutic efficiency of fosfamicin and their pharmaceutical compositions including BHSiO₂, we used experimental sepsis models and a statistical processing methos of the received data (χ²) according to [29, 30].

Microorganisms: Staphylococcus aureus (ATCC No 25923 F-49), Escherichia coli (ATCC No 25922 F-50), Pseudomonas aeruginosa (ATCC N227853 F-51).

Animals: for the experiments we used hybrid mice (CBA×C₅₇Black/₆)CBF₁ according to the “Regulations for test animals use” (USSR Ministry of health order supplement No 755 from 12.08. 1977).

Experimental Sepsis Models

The mice have been injected 0.8 ml of Pseudomonas aeruginosa daily culture suspension with a dosage 5×10⁸ CFU/mouse or Staphylococcus aureus daily culture suspension with a dosage 10¹⁰ CFU/mouse or Escherichia coli daily culture suspension with a dosage 8×10⁸ CFU/mouse. The control group has been injected with 0.8 ml of normal saline solution (0.9% sodium chloride solution). In a day after being infected the test mice have been daily (during 3 days) intravenous injected with 100 mg/kg of antibiotics or different pharmaceutical compositions (antibiotic/BHSiO₂) watered down with 0.25 ml of normal saline solution. The control group of mice has been injected using the same scheme with normal saline solution 0.25 mg.

Antimicrobial therapy efficiency has been estimated basing on the q-ty of the living mice on the 7^{th} day of the experiment [29, 30].

The obtained data stated in Table No 2 reflect the results of 3 independent experiments conducted for each test group.

TABLE NO2
Bacterial sepsis antimicrobial therapy efficiency

| | Mice survival rate on the 7^{th} day of infection* | | | | | |
|---|---|---|---|---|---|---|
| | Staphylococcus | | Escherichia | | Pseudomonas | |
| Test groups | aureus | | coli | | aeruginosa | |
| Normal saline | 0% | (0/30) | 0% | (0/31) | 0% | (0/30) |
| (control) | | | | | | |
| Fosfomycin | 40% | (12/30) | 46.6% | (14/30) | 32.2% | (10/31) |
| Fosfomycin/ | 83.3% | (25/30) | 87% | (27/31) | 76.6% | (23/30) |
| BHSiO₂ (30:1), | P < 0.01** | | P < 0.01** | | P < 0.01** | |
| m/a 2 hours | | | | | | |
| Fosfomycin/ | 80.6% | (25/31) | 87.5% | (28/32) | 75.7% | (25/33) |

| | | | |
|---|---|---|---|
| BHSiO₂ (50:1), | P < 0.01** | P < 0.01** | P < 0.01** |
| m/a 2 hours | | | |

*survival rate/infected animals rate measured in % and absolute values
**comparing to the group of mice having Fosfomycin

As can be seen from Table No 2 the suggested antimicrobial and anti-inflammatory action pharmaceutical composition (fosfomycin/BHSiO₂) definitely possess an increased therapeutic efficiency (2 times higher) comparing to simple fosfomycin in case of lab animals sepsis treatment, provoked by Pseudomonas aeruginosa, Staphylococcus aureus or Escherichia coli.

### Example No 3

### Fosfomycin and Pharmaceutical Composition Anti-Inflammatory Action Determination

It is known that in case of sepsis the quantity of peripheral blood leucocytes can determine the activity of the inflammatory process [31].

We have studied the leukocytes quantity of the mice that have survived until the day 7 after sepsis induction, according to the method described above. The obtained data are shown in Table No 3.

TABLE NO3
Peripheral blood leukocytes quantity of the mice which have
survived until day 7 after being infected with Pseudomonas aeruginosa
and Staphylococcus aureus treated with fosfomycin and pharmaceutical
composition

| Test groups (each group | Leukocytes quantity × 10⁹/l |
|---|---|
| contains not less than 10 mice) | Me(LQ-HQ)* |
| 1. Intact mice. | 8.31 (6.55-9.44) |
| Normal saline (control) | |
| 2. Mice infected with | 17.38 (15.5-21.5) |
| Staphylococcus aureus. | P₁₋₂ < 0.001 |
| Fosfomycin | |
| 3. Mice infected with | 12.25 (11.63-13.75) |
| Staphylococcus aureus. | P₁₋₃ < 0.01 |
| Fosfomycin:BHSiO₂ (30:1), | |
| m/a 2 hours | P₂₋₃ < 0.05 |
| 4. Mice infected with | 17.13 (16.0-18.63) |
| Pseudomonas aeruginosa. | P₁₋₄ < 0.001 |
| Fosfomycin | |
| 5. Mice infected with | 12.14 (11.53-13.0) |
| Pseudomonas aeruginosa. | P₁₋₅ < 0.01 |
| Fosfomycin:BHSiO₂ (30:1), | |
| m/a 2 hours | P₄₋₅ < 0.02 |

*median, low and high quartiles

As may be seen from Table No 3 the proposed pharmaceutical composition (fosfomycin/BHSiO₂) an authentically expressed anti-inflammatory action comparing with fosfomycin in case of test animals sepsis treatment provoked by Pseudomonas aeruginosa or Staphylococcus aureus.

In that case, basing on the received data we may come to the conclusion that the proposed antimicrobial and anti-inflammatory action pharmaceutical composition (fosfomycin/BHSiO₂) has a real increased therapeutic effect in case of treating severe contagious and inflammatory diseases comparing to the initial fosfomycin (prototype of the mentioned invention).

### Used Literature

1. I. B. Mikhailov//Clinical pharmacology, medics table book.—St. Petersburg: [symbol] Foliant [symbol] Publishing, 2001.—736 p.
2. Joukhadar C., Klein N., Dittrich P. et al. Target site penetration of fosfomycin in critically ill patients//J. Antimicrob. Chemother.—2003.—Vol. 51.—P. 1247-1252.
3. Sauermann R., Karch R., Langenberger H. et al. Antibiotic abscess penetration: fosfomycin levels measured in pus and simulated concentration-time profiles//Antimicrob. Agents Chemother.—2005.—Vol. 49.—P. 4448-4454.
4. Schintler M. V., Traunmuller F., Metzler J. et al. High fosfomycin concentrations in bone and peripheral soft tissue in diabetic patients presenting with bacterial foot infection//J. Antimicrob. Chemother.—2009.—Vol. 64.—P. 574-578.
5. Matzi V., Lindenmann J., Porubsky C. et al. Extracellular concentrations of fosfomycin in lung tissue of septic patients//J. Antimicrob. Chemother.—2010.—Vol. 65.—P. 995-998.
6. Kastoris A. C., Rafailidis P. I., Vouloumanou E. K. et al. Synergy of fosfomycin with other antibiotics for Gram-positive and Gram-negative bacteria//Europ. J. Clinical Pharmacology.—2010.—Vol. 66.—P. 359-368.
7. Cai Y., Fan Y., Wang R. et al. Synergistic effects of aminoglicosides and fosfomycin on Pseudomonas aeruginosa in vitro and biofilm infections in a rat model//J. Antimicrob. Chemother.—2009.—Vol. 64.—P. 563-566.
8. Zeitlinger M., Marsik C., Steiner I. et al. Immunomodulatory effects of fosfomycin in an endotoxin model in human blood//J. Antimicrob. Chemother.—2006.—Vol. 59.—P. 219-223.
9. Presterl E., Hajdu S., Lassnigg A. M. et al. Effects of azithromycin in combination with vancomycin, daptomycin, fosfomycin, tigecycline and ceftriaxone on Staphylococcus epidermidis biofilms//Mimicrob. Agents Chtmother.—2009.—Vol. 53.—P. 3205-3210.
10. Abeylath S. C., Turos E. Drug delivery approaches to overcome bacterial resistance to β-lactam antibiotics//Expert Opinion on Drug Delivery.—2008.—Vol. 5.—P. 931-949.
11. Bastus N. G., Sanchez-Tillo E., Pujals S. et al. Peptides conjugated to gold nanoparticles induce macrophage activation//Molecular Immunology.—2009.—Vol. 46.—P. 743-748.
12. Pinto-Alphandary H., Andremont A., Couvreur P. Targeted delivery of antibiotics using liposomes and nanoparticles: research and applications//International Journal of Antimicrobial Agents.—2000.—Vol. 13.—P. 155-168.
13. Ulbrich W., Lamprech A. Targeted drug-delivery approaches by nanoparticulate carriers in the therapy of inflammatory diseases//Journal Royal Society Interface.—2010.—Vol. 7, Suppl. 1.—P.S 55-S66.
14. A. E. Guliaev, B. A. Ermekbaeva, G. Y. Kivman and etc. Nano-particles as antibiotics direct transportation vector (review)//Chemical and pharmaceutical magazine.—1998.—No 3.—P. 3-6.
15. Rosemary M. J., MacLaren I., Pradeep T. Investigation of antibacterial properties of ciprofloxacin@SiO₂.//Langmuir.—2006.—Vol. 22.—P. 10125-10129.
16. Rai A., Prabhune A., Perry C. C. Antibiotic mediated synthesis of gold nanoparticles with potent antimicrobial activity and their application in antimicrobial coatings//Journal of Materials Chemistry.—2010.—Vol. 20.—P. 6789-6798.
17. Zolnik B. S., Gonzalez-Fernandez A., Sadrieh N., Dobrovolskaia V. Minireview: Nanoparticles and the immune system//Endocrinology.—2010.—Vol. 151.—P. 458-465.
18. [symbol] H. A., [symbol] A. H., POMaHOB M. [symbol] ., [symbol] [symbol] C.// [symbol] [symbol] [symbol] - [symbol] [symbol] [symbol] [symbol] [symbol] [symbol] —CapaHCK.: [symbol] -BO [symbol] [symbol] [symbol] [symbol] 2007.—140 c.
19. Pinto-Alphandary H., Balland O., Laurent M. et al. Intracellular visualization of ampicillin-loaded nanoparticles in peritoneal macrophages infected in vitro with Salmonella typhimurium//Pharmaceutical Research.—1994.—Vol. 11.—P. 38-46.
20. Park J-H., Gu L., Maltzahn G. et al. Biodegradable luminescent porous silica nanoparticles for in vivo applications//Nature Materials.—2009.—Vol. 8.—P. 331-336.
21. Pernis B. Silica and the immune system//Acta Biomed.—2005.—Vol. 76, Suppl. 2.-P. 38-44.
22. Tasciotti E., Liu X., Bhavane R. Et et al. Mesoporous silica particles as a multistage delivery system for imaging and therapeutic applications//Nature Nanotechnology.—2008.—Vol. 3.—P. 151-157.
23. Seleem M. N., Munusamy P., Ranjan A et al. Silica-antibiotic hybrid nanoparticles for targeting intracellular pathogens//Antimicrob. Agents Chemother.—2009.—Vol. 53.—P. 4270-4274.
24. Clinical chemistry and silica dioxide clinical use// [symbol] [symbol] [symbol] HAH [symbol] A. A. [symbol] — [symbol] : [symbol] [symbol] , 2003.—416 c.
25. Chuiko A., Pentyuk A., Shtat'ko E., Chuiko N. Medical aspects of application of highly disperse amorphous silica//Surface Chemistry in Biomedical and Environmental Science. Edited by J. P. Blitz and V. Gun'ko.II. Mathematics, Physics and Chemistry.—2006.—Vol. 228.—P. 191-204.
26. Waters K. M., Masiello L. M., Zangar R. C. et al. Macrophage responses to silica nanoparticles are highly conserved across particle sizes//Toxicological Sciences.—2009.—Vol. 107.—P. 553-569.
27. Lucarelli M., Gatti A. M., Savarino G. et al. Innate defence functions of macrophages can be biased by nano-sized ceramic and metallic particles//European Cytokine Network.—2004.—Vol. 15.—P. 339-346.
28. Hamilton R. F., Thakur S. A., Mayfair J. K., Holian A. MARCO mediates silica uptake and toxicity in alveolar macrophages from C57BL/6 mice//J. Biological Chemistry.—2006.—Vol. 281.—P. 34218-34226.
29. Eckhardt C., Fickweiler K., Schaumann R. et al. Therapeutic efficacy of moxifloxacin in a murine model of severe systemic mixed infection with E. coli and B. fragilis//Anaerobe.—2003.—Vol. 9.—P. 157-160.
30. Schaumann R., Blatz R., Beer J. et al. Effect of moxifloxacin versus imipenem/cilastatin treatment on the mortality of mice infected intravenously with different strains of Bacteroides fragilis and Escherichia coli//Journal of Antimicrobial Chemotherapy.—2004.—Vol. 53.—P. 318-324.
31. Aird W. C. The hematologic system as a marker of organ dysfunction in sepsis//Mayo Clin. Proc.—2003.—Vol. 78.—P. 869-881.

1. An antimicrobial pharmaceutical composition for parenteral administration made in the form of injection preparation powder, said composition including: a first amount of fosfomycin acting as a therapeutic agent, and a second amount of finely dispersed nanostructured silica dioxide; wherein said composition is characterized by a weight ratio of the first amount to the second amount, and said weight ratio constituting: (10-75):1.

2. The composition according to claim 1, wherein said finely dispersed nanostructured silica dioxide is composed of particles; said finely dispersed nanostructured silica dioxide includes a portion of at least 25% thereof, wherein said portion substantially consists of said particles having a size not exceeding 5 micrometers.

3. A process of production of an antimicrobial pharmaceutical composition for parenteral administration comprising the steps of:
providing a first amount of fosfomycin in the form of powder;
providing a second amount of finely dispersed nanostructured silica dioxide in the form of powder;
mixing said first amount with said second amount in a weight ratio of (10-75):1, thereby obtaining a mixture; and
subjecting said mixture to mechanical processing by means of blow-abrasive actions.

4. The process according to claim 3, wherein said finely dispersed nanostructured silica dioxide is composed of particles; said blow-abrasive actions are resulted in that said finely dispersed nanostructured silica dioxide includes a portion of at least 25% thereof, wherein said portion substantially consists of said particles having a size not exceeding 5 micrometers.
